(19) United States
(12) Patent Application Publication
(10) Pub. No.: US 2008/0085877 A1
(43) Pub. Date: Apr. 10, 2008

(54) Therapeutic methods of using estrogen compositions

(75) Inventor: Jonathan Bortz, St. Louis, MO (US)

Correspondence Address: KV PHARMACEUTICAL COMPANY, 4080B WEDGEWAY COURT, EARTH CITY, MO, 63045, US

(73) Assignee: DrugTech Corporation, Wilmington, DE (US)

(21) Appl. No.: 11/502,066

(22) Filed: Aug. 10, 2006

(51) Int. Cl. A61K 31/56; A61P 15/00

(52) U.S. Cl. 514/182

(57) ABSTRACT

A method for preventing or treating a catamenial migrainous disorder in a female subject comprises administering to a vulvovaginal surface of the subject a pharmaceutical composition that is bioadhesive thereto and comprises at least one estrogenic compound in an amount of about 50 μg to about 1000 μg estradiol equivalent per unit dose of the composition. A related method comprises administering to a vulvovaginal surface of the subject a pharmaceutical composition comprising at least one estrogenic compound, wherein upon administration of the composition to the vulvovaginal surface, a decline in serum estradiol concentration during a luteal phase of a menstrual cycle is moderated.

This application contains subject matter that is related to concurrently filed U.S. application Ser. No. ______, titled “Estrogen compositions and therapeutic methods of use thereof”, the entire disclosure of which is incorporated by reference herein.

### FIELD OF THE INVENTION

The present invention relates to therapeutic methods of use of pharmaceutical compositions suitable for vaginal delivery of an estrogen compound in women having migraine or headache.

### BACKGROUND OF THE INVENTION

Migraine is the most common form of disabling headache and may well be one of the most common reasons for patients to see their doctors. In 2003, the International Headache Society adopted the new International Classification of Headache Disorders—2nd edition (ICHD-II). Migraine diagnostic criteria according to ICHD-II include recurrent headache disorder manifesting in attacks lasting from 4 to 72 hours; headache with features such as unilateral location, pulsating quality, and moderate or severe pain intensity; aggravation of pain by routine physical activity; and association with nausea, photophobia, and/or phonophobia. Chronic migraine includes these same features but further includes headache on more than 15 days a month. Headache Classification Committee of the International Headache Society (2004) Cephalalgia 24(Supp. 1):1-160.

Menstrual migraine is also classified by ICHD-II with the features as discussed above for general migraine, but this migraine classification is relegated to the Appendix due to uncertainties as to whether pure menstrual migraine and menstrually related migraine should be given separate entries. Although both types of migraine occur on day 1 of the menstrual cycle, plus or minus 2 days (i.e., days −2 to +3) in at least two out of three menstrual cycles, menstrually related migraine can additionally occur at other times of the cycle while pure menstrual migraine is applicable only to women during menses. Id, at page 138.

Estrogen withdrawal migraine is also classified by ICHD-II. ICHD-II mentions that headache or migraine can be induced by estrogen withdrawal following cessation of a course of exogenous estrogens (including the pill-free interval or placebo phase of an oral contraceptive or hormone replacement regimen). The diagnostic criteria include the criteria applicable to general migraine as described above, and additionally, uninterrupted daily use of exogenous estrogen for three or more weeks and migraine that develops within 5 days after last use of estrogen and resolves within 3 days. Id, at page 96.

Initial migraine treatments include administration of analgesics and/or non-steroidal anti-inflammatory drugs (NSAIDs), and when these fail, any one of the triptans (selective serotonin (5-HT) receptor agonists) can be tried. Preventive treatments such as β-blockers, tricyclics and anticonvulsants leave much to be desired with side effects such as sleepiness, exercise intolerability, erectile dysfunction, nightmares, dry mouth, weight gain, tremor, hair loss, or fetal deformities. New drugs are needed to prevent or treat migraine, in particular menstrual migraine, including pure menstrual migraine, menstrually related migraine, and estrogen withdrawal migraine.

High-dose estradiol (100 and 200 μg patches administered twice daily) can improve symptoms of premenstrual syndrome (including irritability, headache, depression, fatigue, breast tenderness, breast swelling, insomnia, change of appetite and concentration difficulties during the two weeks prior to menstruation) by suppressing ovulation, according to Drugs and Therapy Perspectives (2004) 20(1):11-15.

Silberstein (2000) Revue Neurologique 156(Supp. 4):4S30-4S41, mentions that menstrually related migraine is due to withdrawal of estrogen or periodic discontinuation of oral contraceptives and also mentions that treatment with estrogen given premenstrually or perimenstrually can delay onset of migraine.

International Patent Publication No. WO 2004/098517 of Ben-Maimon et al. mentions use of extended cycle regimen (estrogen and progestin) to treat catamenial symptoms, including migraine. The proposed daily treatments last for fifty or more days. The publication further mentions intravaginal administration among other routes of administration.

International Application No. WO 2005/007112 of Bell et al. mentions using contraceptive regimens for non-contraceptive benefits such as reduction in pre-menstrual symptoms, including headache. The publication also mentions use of “unopposed estrogen” for the latter 2 to 10 days of the menstrual cycle. Intravaginal administration of estrogen is mentioned among other routes.

U.S. Pat. No. 6,797,282 to Kafrissen & Taitel mentions daily use of oral progestin and/or estrogen with the potential to treat menstrual migraine. Among other routes, intravaginal administration reportedly causes slow release of actives.

U.S. Pat. No. 4,551,148 to Riley et al. proposes a controlled release system for vaginal drug delivery, comprising unit cells having a nonlipoidal internal phase and a lipoidal continuous external phase. An active agent is present at least in the internal phase.

Thompson & Levinson (2002), Drug Delivery Systems & Sciences 2(1), 17-19, describe the VagiSite® bioadhesive topical drug delivery system as a high internal phase ratio water-in-oil emulsion system, providing a delivery platform for administration of active drug entities in the vaginal cavity. They disclose that the VagiSite® system is incorporated in Gynazole-1® antifungal vaginal cream.

U.S. Patent Application Publication No. 2003/0180366 of Kirschner et al. proposes a vaginal drug delivery system having globules comprising an internal water-soluble phase that is acid buffered and contains a drug, and an external water-insoluble phase or film.

U.S. Patent Application Publication No. 2004/0234606 of Levine et al. proposes a composition for vaginal administration comprising a treating agent (the tocolytic drug terbutaline is exemplified) and a bioadhesive cross-linked water-swellable but water-insoluble polycarboxylic acid such as polycarbophil, designed to give controlled and prolonged release of the drug through the vaginal mucosa. Administration of the composition is said to achieve local tissue concentrations without detrimental blood levels.

International Application No. WO 98/20917 of Shah mentions use of a hydrophilic polystyrene graft copolymer delivery vehicle for intravaginal administration of active agents such as progesterone or estrogen.

### SUMMARY OF THE INVENTION

There is now provided a method for preventing or treating a catamenial migrainous disorder, the method comprising administering to a vulvovaginal surface a pharmaceutical composition that is bioadhesive thereto and comprises at least one estrogenic compound in an amount of about 50 μg to about 1000 μg estradiol equivalent per unit dose of the composition.

There is further provided a method for preventing or treating a catamenial migrainous disorder, the method comprising administering to a vulvovaginal surface a pharmaceutical composition comprising at least one estrogenic compound, wherein upon application of the composition to the vulvovaginal surface, a decline in serum estradiol concentration during a luteal phase of a menstrual cycle is moderated.

There is still further provided a method for managing serum estradiol concentration during a menstrual cycle in a female subject, the method comprising a regimen that comprises:
(a) a first regimen component wherein at least one estrogenic compound is administered to the subject according to a first mode of administration; said first mode of administration resulting in maintenance of a serum estradiol concentration of at least about 25 pg/ml during most or all of the menstrual cycle, except for an estrogen depleting phase of about 5 to about 10 days in duration beginning not earlier than about 7 days prior to menses; and
(b) a second regimen component wherein at least one estrogenic compound is administered to the subject according to a second mode of administration; said second mode of administration (i) comprising administering to a vulvovaginal surface a pharmaceutical composition that is bioadhesive thereto and comprises said at least one estrogenic compound; and (ii) resulting in moderation of a decline in serum estradiol concentration during said estrogen depleting phase.

According to any of the above methods, the pharmaceutical composition in one embodiment has at least one non-lipoidal internal phase and at least one lipoidal external phase that is bioadhesive to the vulvovaginal surface; and upon application of the composition to the vulvovaginal surface the at least one estrogenic compound is released over a period of about 3 hours to about 14 days. Illustratively, the composition is a vaginal cream that is bioadhesive to a vaginal mucosal surface and is administered to such a surface.

There is also provided a kit useful for managing serum estradiol concentration during a menstrual cycle in a female subject, the kit comprising:
(a) a first regimen component wherein at least one estrogenic compound is administered to the subject according to a first mode of administration; said first mode of administration resulting in maintenance of a serum estradiol concentration of at least about 25 pg/ml during most or all of the menstrual cycle, except for an estrogen depleting phase of about 5 to about 10 days in duration beginning not earlier than about 7 days prior to menses; and
(b) a second regimen component wherein at least one estrogenic compound is administered to the subject according to a second mode of administration; said second mode of administration (i) comprising administering to a vulvovaginal surface a pharmaceutical composition that is bioadhesive thereto and comprises said at least one estrogenic compound; and (ii) resulting in moderation of a decline in serum estradiol concentration during said estrogen depleting phase.

Additional embodiments are described in the detailed description that follows.

### DETAILED DESCRIPTION

The term “migrainous disorder” herein refers to a disorder resembling, or of the nature of, migraine. A migraine can be a brain disorder involving abnormal sensory processing. A migrainous disorder herein thus includes migraine and all its symptoms, such as headache and cluster headache with moderate or severe pain, nausea, photophobia, and/or phonophobia, and further includes such symptoms even in absence of a specific diagnosis of migraine. The term “catamenial migrainous disorder” herein denotes any migrainous disorder of or relating to the menstrual cycle, menstruation or the menses, or to estrogen withdrawal in pre-, peri- and post-menopausal women. Particular types of migraine such as pure menstrual migraine, menstrually related migraine, and estrogen withdrawal migraine, as well as menstrual headache even where migraine has not been diagnosed, are considered herein to be catamenial migrainous disorders.

Without being bound by theory, it is believed that migrainous disorders can be triggered by a precipitous drop in serum estradiol levels. Serum estradiol levels fluctuate throughout the menstrual cycle, rising gradually during the follicular phase (phase beginning around day 1 of the menstrual cycle), then falling immediately post-ovulation. A secondary rise occurs during the luteal phase (phase beginning post-ovulation and covering the latter half of the menstrual cycle) but a sharp decline typically occurs prior to initiation of menses. The fluctuation of serum estradiol levels can occur as a result of the natural menstrual cycle or can be artificially created or modulated by administration of a hormonal preparation, as for example in use of a hormonal contraceptive or in hormone replacement therapy. Serum estradiol levels can also fluctuate due to other types of hormonal change such as in pregnancy, post-partum and perimenopause.

Hutchinson (2005) Clinics in Family Practice 7(3:Special Issue):529-543, while not admitted to be prior art to the present invention, mentions trying to maintain an even estradiol level to help with hormonally related migraine headaches in women.

In one embodiment, a method of the invention for preventing or treating a catamenial migrainous disorder in a female subject comprises administering to a vulvovaginal surface, e.g., a vaginal mucosal surface, of the subject a pharmaceutical composition that is bioadhesive to such a surface, and comprises at least one estrogenic compound. The at least one estrogenic compound is present in an amount of about 50 μg to about 1000 μg estradiol equivalent per unit dose of the composition.

It can be desirable to maintain at least a baseline estradiol level in the serum throughout the menstrual cycle or to prevent a precipitous decline in serum estradiol levels at one or more points in the menstrual cycle, especially during the luteal phase.

Accordingly, in another embodiment, a method of the invention for preventing or treating a catamenial migrainous disorder in a female subject comprises administering to a vulvovaginal surface of the subject a pharmaceutical composition comprising at least one estrogenic compound, wherein upon application of the composition to the vulvovaginal surface, a decline in serum estradiol concentration during a luteal phase of a menstrual cycle is moderated. Illustratively, the decline that is moderated occurs within about 3 days prior to, or within about 5 days after, onset of menses.

“Moderation” of a decline in serum estradiol concentration herein includes making the decline more gradual, less profound, or both. In some situations, the decline can be substantially eliminated. Where it is desired to maintain at least a baseline serum estradiol concentration, a suitable baseline is typically at least about 25 pg/ml. A “baseline” concentration herein means a minimum concentration consistent with an acceptably low risk or incidence of catamenial migrainous disorder in a particular subject. However, in at least some subjects, serum estradiol concentration can be allowed to decline below a typical baseline without provoking a catamenial migrainous disorder, so long as the decline is gradual.

The decline in serum estradiol concentration can be moderated through partial replacement of endogenous or exogenous estrogen supply. Illustratively, this can be accomplished by (a) initial release of estrogen from the composition to compensate for partial or complete shut-down of endogenous estrogen production or withdrawal of exogenous estrogen; and by (b) extended release of estrogen from the composition, e.g., according to first order kinetics, or (c) follow-up administration at reduced (e.g., progressively reducing) dosage.

A “vulvovaginal surface” herein denotes any external or internal surface of the female genitalia, including mucosal surfaces in the vaginal cavity and nonmucosal surfaces of the vulva and immediately surrounding areas of skin. In some embodiments, the composition is more specifically adapted for application to a vaginal mucosal surface, and is bioadhesive to such a surface.

The composition is typically adapted for release of the at least one estrogenic compound over a period of about 3 hours to about 14 days. In various embodiments, the composition is adapted for release of the at least one estrogenic compound over a period consistent with a once daily to once weekly schedule, or a once to three times per week dosing schedule. In other embodiments, the composition is adapted for release of the at least one estrogenic compound over a period consistent with a single application during the luteal phase, especially during the second half of the luteal phase, of a menstrual cycle.

In particular embodiments, the composition is adapted for release of the at least one estrogenic compound over a period of about 3 hours to about 10 days, for example about 6 hours to about 7 days, about 12 hours to about 5 days, or about 1 to about 3 days.

A “release period” or equivalent phrase herein refers to a period during which the at least one estrogenic compound is made available for absorption at the site of administration, for example the vaginal cavity, in an amount sufficient to provide therapeutic benefit. Thus the “release period” begins when release substantially begins (e.g., immediately to about 1 hour after administration, or later in the case of a delayed-release composition), and ends when substantially no further active agent is available for release (e.g., about 3 hours to about 10 days after the beginning of the release period).

In some embodiments, a method of the invention results in the serum estradiol concentration being maintained at least at a baseline level during the luteal phase, for example substantially the entire luteal phase or more particularly during the second half of the luteal phase, of the menstrual cycle. This can be accomplished by selecting an appropriate dose and release profile of the at least one estrogenic compound. Illustratively, a dose and release profile of the at least one estrogenic compound can be selected to maintain serum estradiol concentration at or above a baseline of at least about 25 pg/ml, at least about 30 pg/ml, at least about 40 pg/ml, or at least about 50 pg/ml.

On the other hand, while it can be desirable to maintain serum estradiol concentration above a certain baseline, it can further be desirable to keep serum estradiol concentration below a certain maximum at the same time. For example, the dose and release profile of the at least one estrogenic compound can be selected to result in a serum estradiol concentration not greater than about 150 pg/ml, not greater than about 125 pg/ml, not greater than about 100 pg/ml, not greater than about 80 pg/ml, or not greater than about 70 pg/ml.

In some embodiments, the composition is administered during a luteal phase of a menstrual cycle. For example, the composition can be administered not earlier than about 10 days prior to menses, or not earlier than about 7 days prior to menses. Such a timing of administration can be especially effective in moderating the often precipitous decline in serum estradiol concentration that occurs shortly before menses and that is believed to be conducive to such conditions as pure menstrual migraine and menstrual headache. Illustratively, the composition can be administered during the placebo phase of an oral contraceptive regimen.

In other embodiments, the composition is administered during menses. In such embodiments, the bioadhesiveness of the composition provides a significant benefit in reducing or preventing loss of the composition by flushing out with the flow of menstrual fluid. Thus in an important embodiment, the composition exhibits sufficient bioadhesion to the vulvovaginal surface to substantially resist elution by menstrual flow.

In still other embodiments, the composition is administered during a luteal phase of a menstrual cycle and again during menses. A suitable regimen of administration for a particular subject can be determined by a prescribing physician, and can be based, for example, on the responsiveness to treatment and/or the timing of specific migraine triggers.

A composition useful herein comprises at least one estrogenic compound. An “estrogenic compound” herein is any compound or mixture thereof, whether of natural, biosynthetic or chemosynthetic origin, having estrogenic activity in a human female. Estrogenic compounds include steroidal and nonsteroidal compounds. Illustrative nonsteroidal estrogenic compounds include without limitation broparoestrol, chlorotrianisene, dienestrol, diethylstilbestrol, fosfestrol, hexestrol, methestrol, derivatives such as salts and esters thereof, enantiomers and racemates thereof, mixtures thereof and the like. Illustrative steroidal estrogenic compounds include without limitation conjugated estrogenic hormones (e.g., Premarin®), equilenin, equilin, estradiol, estriol, estrone, ethinyl estradiol, mestranol, moxestrol, quinestradiol, quinestrol, derivatives such as salts and esters thereof, enantiomers and racemates thereof, mixtures thereof and the like.

In an illustrative embodiment the at least one estrogenic compound is a steroid.

In a more particular illustrative embodiment the at least one estrogenic compound comprises a compound selected from the group consisting of conjugated estrogenic hormones, estradiol, ethinyl estradiol, estriol and estrone.

In a still more particular illustrative embodiment the at least one estrogenic compound comprises estradiol or a derivative thereof, e.g., ethinyl estradiol.

Amounts of the at least one estrogenic compound are expressed herein as estradiol equivalent amounts unless the context demands otherwise. In a particular low-dose embodiment, a composition of the invention provides about 50 to about 500 μg, for example about 100 to about 500 μg, about 150 to about 500 μg, or about 200 to about 500 μg, estradiol equivalent per unit dose. In various embodiments the at least one estrogenic compound is present in a total estradiol equivalent amount of about 200 to about 450 μg, about 200 to about 400 μg, about 250 to about 400 μg, about 300 to about 400 μg or, illustratively, about 50 μg, about 100 μg, about 150 μg, about 200 μg, about 250 μg, about 300 μg, about 350 μg, about 400 μg, about 450 μg or about 500 μg per unit dose of the composition.

A unit dose is an amount of the composition suitable for a single administration to a vulvovaginal surface, for example a vaginal mucosal surface, as described herein. Most conveniently for the patient, the composition is provided in unit dose aliquots, typically individually packaged, but this is not a requirement of the present invention. A convenient unit dose aliquot of a vaginal cream is an amount of about 1 to about 10 g, although greater or lesser amounts, for example as little as about 0.1 g or as much as about 25 g, can be used if desired. A particularly suitable unit dosage amount of a vaginal cream is about 2 to about 6 g, for example about 2 g, about 3 g, about 4 g or about 5 g. Where a unit dose is an amount of about 5 g, the total estradiol equivalent concentration in a low-dose composition is suitably about 10 to about 100 μg/g, for example about 20 to about 100 μg/g, about 30 to about 100 μg/g, or about 40 to about 100 μg; in various embodiments about 40 to about 90 μg/g, about 40 to about 80 μg/g, about 50 to about 80 μg/g, about 60 to about 80 μg/g, or, illustratively, about 10 μg/g, about 20 μg/g, about 30 μg/g, about 40 μg/g, about 50 μg/g, about 60 μg/g, about 70 μg/g, about 80 μg/g, about 90 μg/g or about 100 μg/g. Where the unit dose is greater or smaller than 5 g, suitable estradiol equivalent concentration ranges will be correspondingly lower or higher respectively.

In some embodiments, the amount of the at least one estrogenic compound is modulated to compensate for reduced absorption resulting from premenopausal thickness of the vaginal epithelium. In this embodiment, a high-dose composition can be used.

In a particular high-dose embodiment, the estradiol equivalent amount in the composition is about 350 to about 1000 μg, for example about 350 to about 750 μg, about 400 to about 500 μg, or about 400 to about 450 μg per unit dose. For some uses, a cream having an estradiol equivalent concentration substantially higher than about 400 μg per unit dose, for example about 420, about 425, about 440, about 450, about 460 or about 475 μg per unit dose, can be provided.

The pharmaceutical composition can be in any form adapted for application to a vulvovaginal surface, including for example a vaginal cream, thermally gelling formulation, tablet, pessary or implant. In a particular embodiment, the composition is in the form of a vaginal cream.

A particular composition useful according to a method of the invention can illustratively comprise a water-in-oil emulsion as generally described in any of above-referenced U.S. Pat. No. 4,551,148, U.S. Pat. No. 5,266,329 or U.S. Patent Application Publication No. 2003/0180366, or as further described in U.S. Patent Application No. 2005/0095245 of Riley et al., but differs from these at least in that it comprises an estrogenic compound as active agent. Such a water-in-oil emulsion can be presented in a semi-solid form, for example as a vaginal cream as indicated above.

In a particular embodiment, the pharmaceutical composition is a vaginal cream having at least one nonlipoidal internal phase and at least one lipoidal external phase that is bioadhesive to a vaginal mucosal surface.

Such a composition can be formulated, for example, as the VagiSite® bioadhesive topical drug delivery system described by Thompson & Levinson (2002), op. cit., or a delivery system substantially equivalent thereto, with inclusion of at least one estrogenic compound as active agent.

Conveniently, a unit dosage amount of a vaginal cream used according to the method can be furnished in a prefilled container or applicator, for example an applicator similar to that used for Gynazole-1® vaginal cream of KV Pharmaceutical Co., St Louis, Mo. The applicator can be disposable, and more particularly, the applicator can be prefilled with a unit dose of the composition.

The at least one estrogenic compound can be present in either one or both of the internal and external phases. In one embodiment the at least one estrogenic compound is present at least in part in the internal phase of the composition, and can be in dispersed form, for example in solution or suspension therein, or in non-dispersed form. Optionally, substantially all of the at least one estrogenic compound can be present in the internal phase. Solubilization of the at least one estrogenic compound can be achieved, for example, by use of a cosolvent and/or surfactant. The at least one estrogenic compound can be present at least in part in particulate form, for example in micronized form, and can be dispersed as a particulate suspension in the internal and/or external phase. In various embodiments the at least one estrogenic compound is present in solution, in aggregates, in liposomes, in microcapsules and/or in micelles within the internal and/or external phase. If present in both internal (nonlipoidal) and external (lipoidal) phases, the at least one estrogenic compound can be present in similar or different amounts in the nonlipoidal and lipoidal phases.

As discussed above with respect to compositions in general used according to the method, the composition is adapted to release the at least one estrogenic compound over a period of about 3 hours to about 14 days, upon application to a vulvovaginal surface, for example a vaginal mucosal surface. Based on the disclosure herein, including disclosure of documents incorporated by reference herein, in particular above-referenced U.S. Pat. Nos. 4,551,148 and 5,266,329, and U.S. Patent Application Publication Nos. 2003/0180366 and 2005/0095245, one of skill in the art can without undue experimentation adjust release rate of the at least one estrogenic compound from the composition to achieve a desired release period as set forth herein.

Release rate can be determined by in vivo testing or by any suitable in vitro method. An illustrative in vitro method utilizes an open chamber diffusion cell system such as a Franz cell system, typically fitted with an appropriate inert synthetic membrane such as polysulfone, cellulose acetate/nitrate mixed ester or polytetrafluoroethylene of suitable thickness, e.g., 70 μm. The receptor medium should be one in which the estrogenic compound of interest is soluble, for example a water/ethanol medium. A test composition is placed uniformly on the membrane (illustratively, about 300 mg of a semi-solid composition such as a cream is a suitable amount for placement on a 25 mm diameter membrane) and is kept occluded to prevent solvent evaporation and compositional changes. This corresponds to an infinite dose condition. An aliquot of the receptor fluid is removed for analysis at appropriate intervals, and is replaced with an aliquot of fresh receptor fluid, so that the membrane remains in contact with the receptor fluid throughout the period of the release study. A release rate study such as that outlined above is typically replicated and can be conducted using a standard composition having known release properties for comparison.

The composition typically comprises unit cells each having internal and external phases. The at least one internal phase can be discontinuous and is nonlipoidal and generally miscible with water. Illustratively, the internal phase comprises water, glycerin, propylene glycol, sorbitol or a combination of two or more thereof. The internal phase can itself be monophasic, biphasic or multiphasic, taking the form for example of a solution, suspension, emulsion or combination thereof. The internal phase can comprise one or more suspended solids, osmotic agents, extenders, diluents, buffers, chelating agents, preservatives or other materials. Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or is detrimental to common pathogens of the vaginal cavity, including fungal pathogens such as Candida species and bacterial pathogens such as Enterococcus species. Typically such a pH is approximately 4.5.

The external phase is lipoidal and generally continuous. The term “lipoidal” herein can pertain to any of a group of organic compounds including neutral fats, fatty acids, waxes, phosphatides, petrolatum, fatty acid esters of monoprotic alcohols, mineral oils, etc., having the following properties: insoluble in water; soluble in alcohol, ether, chloroform or other fat solvents; and exhibiting a greasy feel. Examples of suitable oils are mineral oils having viscosity of about 5.6 to about 68.7 centistokes, for example about 25 to about 65 centistokes, and vegetable oils such as coconut, palm kernel, cocoa butter, cottonseed, peanut, olive, palm, sunflower, sesame, corn, safflower, rapeseed (canola) and soybean oils and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoylphosphatidylcholine, dipentadecanoylphosphatidylcholine, dipalmitoylphosphatidylcholine (DPPC) and distearoylphosphatidylcholine (DSPC); phosphatidylethanolamine esters such as dioleoylphosphatidylethanolamine and dipalmitoylphosphatidylethanolamine (DPPE); phosphatidylserine; phosphatidylglycerol; phosphatidylinositol; etc. Phospholipids and other amphiphilic compounds can enhance stability of the present compositions.

Amphiphilic compounds can act as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, and polyglyceryl esters of fatty acids, such as polyglyceryl-3 oleate. Such agents can also function as emollients in the composition.

Factors affecting release rate of the estrogenic compound(s) can include the particular estrogenic compound(s) used, the physical form of the estrogenic compound(s) (e.g., whether in solution or in particulate form, and if particulate, average particle size), viscosity of the composition, selection and relative amounts of lipoidal compounds, including amphiphilic compounds, in the external phase, osmotic properties of the internal phase, and the relative volumes of the internal and external phases, among other factors. In a composition having the estrogenic compound(s) in the internal phase, and having a relatively small internal phase ratio (expressed as percentage of total volume occupied by the internal phase), the external phase tends to form a relatively thick membrane through which the estrogenic compound(s) must pass to be released; accordingly release rate can be significantly slowed in such a composition.

A suitable internal phase ratio can be established for any particular system by routine testing. In one embodiment the internal phase ratio is at least about 70% by volume.

A semi-solid composition used according to methods of the invention, such as a vaginal cream, can have a viscosity of about 5,000 to about 1,000,000 centipoise, for example about 5,000 to about 750,000 centipoise, about 100,000 to about 800,000 centipoise, about 100,000 to about 400,000 centipoise, about 350,000 to about 750,000 centipoise, about 100,000 to about 550,000 centipoise, about 250,000 to about 400,000 centipoise, about 200,000 to about 350,000 centipoise, or about 350,000 to about 550,000 centipoise. Bioadherence of a composition to a mucosal surface requires, among other properties, sufficient viscosity to retain integrity of the composition. Optional ingredients that can increase viscosity, among other properties, include microcrystalline wax, colloidal silicon dioxide, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like. In an illustrative embodiment, a composition useful herein is a thermally gelling formulation comprising a thermosetting polymer, e.g., a poloxamer such as poloxamer 407 (available, for example, as Pluronic™ F-127 of BASF).

In an illustrative embodiment, a vaginal cream comprises at least one estrogenic compound, for example estradiol, ethinyl estradiol or estrone, water, sorbitol, propylene glycol, at least one long chain monoglyceride, for example glyceryl monooleate, glyceryl monostearate, glyceryl monoisostearate or glyceryl monopalmitate, a chelating agent, for example edetate disodium, at least one antimicrobial preservative, for example methylparaben and/or propylparaben, mineral oil, microcrystalline wax and colloidal silicon dioxide, for example hydrophobically modified colloidal silicon dioxide.

A composition, used according to a method of the invention, in the form of a vaginal cream can be prepared by known batch or continuous processes for preparing pharmaceutical creams. As in preparing conventional emulsions, shear force is applied to the components by use of a mixer, homogenizer, mill, impingement surface, ultrasound, shaking or vibration. Mixing shear should be at a relatively low level to prevent destruction of the emulsion by excess energy.

Illustratively, the internal and external phases are first prepared separately. In a typical batch process, the internal phase is added to the external phase while mixing in a planetary-type or other suitable mixer until a stable emulsion is formed. Addition rates and mixing speeds can be adjusted to optimize formation and viscosity of the emulsion. In a typical continuous process, the external phase is introduced into a continuous mixer that comprises a plurality of impellers, until it reaches the level of the lowest impeller in the mixing chamber. The two phases are then simultaneously introduced through the bottom of the mixer in proper proportion as the impellers rotate to apply shear to the components. The finished emulsion emerges through the top of the mixer. Flow rate through the mixing chamber and mixing speed can be adjusted to optimize formation and viscosity of the emulsion.

According to the methods of the invention, the composition can be administered topically to external surfaces of the vulva and/or to surrounding areas of skin. In addition or alternatively, the composition can be administered intravaginally. In one embodiment, the composition is a vaginal cream, i.e., a semi-solid formulation adapted for administration to vaginal mucosal surfaces.

The amount of the composition to be administered will depend on the particular estrogenic compound or compounds present, the concentration of such compound or compounds in the composition, the frequency of administration (as determined for example by release rate) and other factors. For example, to allow for greater thickness of the vaginal epithelium, the amount can be increased to deliver desired plasma levels.

A vaginal cream of the invention can be administered to contact a mucosal surface in the vaginal cavity by means, for example, of an applicator that is optionally pre-filled with a single unit dosage amount of the cream. With the patient optionally in a supine position, the tip of the applicator can be gently inserted high in the vagina, for example in the posterior vaginal fornix, and the cream can be released through the tip by pushing on a plunger of the applicator.

A prolonged release period is enabled by compositions of some embodiments of the invention. Such a prolonged release period brings a number of benefits to the patient, including without limitation those discussed immediately below.

First, frequency of application can be significantly reduced by comparison with a composition having faster release. In general, frequency of application of a prolonged-release composition for effective treatment is once every 2 to 14 days, for example about 1 to about 3 times per week, illustratively about three times weekly, about twice weekly or about once weekly. In some embodiments, frequency of application is once per menstrual cycle, i.e., typically about once per month.

Second, the slow release from such a composition can result in controlled serum levels of estradiol. This minimizes serum fluctuations observed with other dosing regimens such as once daily dosing.

Third, dosage amounts of the estrogenic compound can be reduced to levels close to the lowest effective dose for prevention or treatment of a migrainous disorder, taking advantage of the drug-sparing effect of slow release and minimizing adverse side effects.

In an embodiment of the present invention, a method for managing serum estradiol concentration during a menstrual cycle in a female subject comprises administering at least one estrogenic compound according to a regimen having two components.

In the first regimen component, at least one estrogenic compound is administered to the subject according to a first mode of administration that results in maintenance of a serum estradiol concentration of at least about 50 pg/ml, for example at least about 75 pg/ml, during most or all of the menstrual cycle, except for an estrogen depleting phase of about 5 to about 10 days in duration beginning not earlier than about 7 days prior to menses. The first regimen component can be delivered by any one or more routes, including orally, transdermally, parenterally, subcutaneously, intravenously, intramuscularly, intraperitoneally, transmucosally, buccally, sublingually, intravaginally, by inhalation, by depot injection, or by implantation.

In some embodiments, the first regimen component can comprise any element of a hormone replacement or contraceptive regimen, including an entire such regimen. Hormone replacement regimens include therapy with estrogen alone, optionally in combination with progestin (including progesterone) and/or testosterone. Any frequency of administration can be utilized with the invention, including sequentially or continuously combined hormone replacement therapy. Contraceptive regimens can include any combined estrogen and progestin regimen, administered for example in the form of oral contraceptives, transdermal patches, vaginal rings or monthly injections.

In the second regimen component, at least one estrogenic compound is administered to the subject according to a second mode of administration that (i) comprises administering to a vulvovaginal surface a pharmaceutical composition that is bioadhesive to such a surface and comprises the at least one estrogenic compound; and (ii) results in moderation of a decline in serum estradiol concentration during the estrogen depleting phase of the first regimen component.

The at least one estrogen compound administered according to the second regimen component can be the same as or different from the at least one estrogen compound administered according to the first regimen component.

The bioadhesive composition and method of administration thereof to a vulvovaginal surface according to the second regimen component can be any such composition and method disclosed herein. Illustratively, the composition is a vaginal cream having at least one nonlipoidal internal phase and at least one lipoidal external phase that is bioadhesive to a vaginal mucosal surface. The at least one estrogenic compound is present in the cream in an amount typically of about 50 to about 500 μg estradiol equivalent per unit dose of the cream, and, upon application of the cream to the vaginal mucosal surface, is released over a period of about 3 hours to about 14 days. The at least one estrogenic compound can be present in the nonlipoidal phase, the lipoidal phase or both; similar or different amounts can be present in the nonlipoidal and lipoidal phases.

In a related embodiment of the present invention, a kit useful for managing serum estradiol concentration during a menstrual cycle in a female subject comprises (a) one to a plurality of compositions adapted for use in a regimen that results in maintenance of a serum estradiol concentration of at least about 50 pg/ml, for example at least about 75 pg/ml, during most or all of the menstrual cycle, except for an estrogen depleting phase of about 5 to about 10 days in duration beginning not earlier than about 7 days prior to menses; and (b) a pharmaceutical composition that is bioadhesive to a vulvovaginal surface and comprises at least one estrogenic compound in an amount of about 50 to about 500 μg estradiol equivalent per unit dose of the composition.

The kit optionally further comprises instructions, in hard-copy and/or electronic form, instructions for vulvovaginal administration of the bioadhesive composition to prevent or treat a catamenial migrainous disorder promoted by the regimen.

The bioadhesive composition in such a kit can be any such composition disclosed herein. Illustratively, the composition is a vaginal cream having at least one nonlipoidal internal phase and at least one lipoidal external phase that is bioadhesive to a vaginal mucosal surface. The at least one estrogenic compound is present in the cream in an amount typically of about 50 to about 500 μg estradiol equivalent per unit dose of the cream, and, upon application of the cream to the vaginal mucosal surface, is released over a period of about 3 hours to about 14 days.

In one embodiment, a kit as described above further comprises an ovulation indicator, i.e., a device or material that detects timing of ovulation, from which a date of expected onset of menses (and therefore a date of expected onset of an estrogen depleting phase) can be predicted. Any ovulation indicator known in the art, for example a test strip for use with a biofluid such as saliva or urine, can be used. Instructions optionally included as part of the kit can provide direction on use of the ovulation indicator in conjunction with vulvovaginal administration of the bioadhesive composition to prevent or treat a catamenial migrainous disorder.

### EXAMPLES

The following example is merely illustrative, and does not limit this disclosure in any way.

### Example 1

Vaginal cream formulations (Table 1) containing estradiol at 50-500 μg/g can be prepared by any method known in the art for preparing semi-solid emulsions, including batch and continuous processes as described hereinabove.

“USP” refers to U.S. Pharmacopeia; “NF” refers to National Formulary.

Compositions of Table 1 can be administered in a dosage amount of about 5 g to a vulvovaginal surface for treatment of a catamenial migrainous disorder, for example menstrual migraine, according to a method as described herein.

TABLE 1
Estradiol vaginal cream formulations

| | Wt % | | | | |
|---|---|---|---|---|---|
| | Estradiol (target μg/g): | | | | |
| Ingredient | 10 | 30 | 50 | 70 | 100 |
| water, purified, USP | 39.819 | 39.817 | 39.815 | 39.813 | 39.81 |
| sorbitol solution, USP | 40.000 | 40.000 | 40.000 | 40.000 | 40.00 |
| propylene glycol, USP | 5.000 | 5.000 | 5.000 | 5.000 | 5.00 |
| edetate disodium, USP | 0.050 | 0.050 | 0.050 | 0.050 | 0.05 |
| estradiol, USP | 0.001 | 0.003 | 0.005 | 0.007 | 0.01 |
| mineral oil, USP | 8.000 | 8.000 | 8.000 | 8.000 | 8.00 |
| polyglyceryl-3-oleate | 2.750 | 2.750 | 2.750 | 2.750 | 2.75 |
| glyceryl monoisostearate | 2.750 | 2.750 | 2.750 | 2.750 | 2.75 |
| microcrystalline wax, NF | 0.400 | 0.400 | 0.400 | 0.400 | 0.40 |
| silicon dioxide, | 1.000 | 1.000 | 1.000 | 1.000 | 1.00 |
| hydrophobic | | | | | |
| methylparaben, NF | 0.180 | 0.180 | 0.180 | 0.180 | 0.18 |
| propylparaben, NF | 0.050 | 0.050 | 0.050 | 0.050 | 0.05 |

All patents and publications cited herein are incorporated by reference into this application in their entirety.

The words “comprise”, “comprises”, and “comprising” are to be interpreted inclusively rather than exclusively.

What is claimed is:

1. A method for preventing or treating a catamenial migrainous disorder in a female subject, the method comprising administering to a vulvovaginal surface of the subject a pharmaceutical composition that is bioadhesive thereto and comprises at least one estrogenic compound in an amount of about 50 μg to about 1000 μg estradiol equivalent per unit dose of the composition.

2. The method of claim 1, wherein the at least one estrogenic compound is present in the composition in an amount of about 150 μg to about 500 μg per unit dose.

3. The method of claim 1, wherein the composition has at least one non-lipoidal internal phase and at least one lipoidal external phase that is bioadhesive to the vulvovaginal surface.

4. The method of claim 1, wherein the composition releases the at least one estrogenic compound according to a release profile effective to moderate a decline in serum estradiol concentration.

5. The method of claim 1, wherein the composition releases the at least one estrogenic compound according to a release profile consistent with a once daily to once weekly schedule.

6. The method of claim 1, wherein the composition releases the at least one estrogenic compound according to a release profile consistent with a once to three times per week dosing schedule.

7. The method of claim 1, wherein the composition is administered at a frequency of about 1 to about 3 times per week.

8. The method of claim 1, wherein upon application of the composition to the vulvovaginal surface the at least one estrogenic compound is released over a period of about 3 hours to about 14 days.

9. The method of claim 8, wherein a serum estradiol concentration not greater than about 70 pg/ml is maintained in the subject during the release period.

10. The method of claim 1, wherein the composition is administered not more than about 7 days prior to anticipated menses.

11. The method of claim 10, wherein at least a baseline serum estradiol concentration is maintained in the subject from the time of administration through menses.

12. The method of claim 11, wherein the baseline serum estradiol concentration is not less than about 25 pg/ml.

13. The method of 11, wherein the baseline serum estradiol concentration is not less than about 50 pg/ml.

14. The method of claim 1, wherein the composition is administered during menses.

15. The method of claim 14, wherein the composition exhibits sufficient bioadhesion to the vulvovaginal surface to substantially resist elution by menstrual flow.

16. The method of claim 1, wherein the at least one estrogenic compound is a steroid.

17. The method of claim 1, wherein the at least one estrogenic compound is selected from the group consisting of conjugated estrogenic hormones, estradiol, ethinyl estradiol, estriol and estrone.

18. The method of claim 1, wherein the at least one estrogenic compound is estradiol or ethinyl estradiol.

19. The method of claim 1, wherein the vulvovaginal surface to which the composition is adapted for application is a vaginal mucosal surface.

20. The method of claim 19, wherein the composition is in a form of a vaginal cream.

21. The method of claim 20, wherein the vaginal cream is administered with the aid of an applicator.

22. The method of claim 21, wherein the applicator is disposable.

23. The method of claim 22, wherein the applicator is prefilled with a unit dose amount of the vaginal cream.

24. The method of claim 20, wherein the vaginal cream has at least one nonlipoidal internal phase and at least one lipoidal external phase that is bioadhesive to the vaginal mucosal surface, and wherein upon application of the composition thereto the at least one estrogenic compound is released over a period of about 3 hours to about 14 days.

25. The method of claim 1, wherein the catamenial migrainous disorder is selected from the group consisting of pure menstrual migraine, menstrually related migraine, menstrual headache and estrogen withdrawal migraine.

26. A method for preventing or treating a catamenial migrainous disorder in a female subject, the method comprising administering to a vulvovaginal surface of the subject a pharmaceutical composition comprising at least one estrogenic compound, wherein upon application of the composition to the vulvovaginal surface, a decline in serum estradiol concentration during a luteal phase of a menstrual cycle is moderated.

27. The method of claim 26, wherein the decline that is moderated occurs during the second half of the luteal phase.

28. The method of claim 26, wherein the decline that is moderated occurs within about 3 days prior to, or within about 5 days after, onset of menses.

29. The method of claim 26, wherein the decline that is moderated occurs during an estrogen depleting phase of hormonal therapy or contraception.

30. The method of claim 26, wherein the amount of the at least one estrogenic compound administered and/or the release rate of the at least one estrogenic compound from the composition is modulated to compensate for differences in absorption resulting from differing thickness of vaginal epithelium.

31. The method of claim 26, wherein the pharmaceutical composition is a vaginal cream having at least one nonlipoidal internal phase and at least one lipoidal external phase that is bioadhesive to a vaginal mucosal surface, wherein the at least one estrogenic compound is present in an amount of about 50 μg to about 1000 μg estradiol equivalent per unit dose of the vaginal cream, and wherein upon application of the composition to the vaginal mucosal surface the at least one estrogenic compound is released over a period of about 3 hours to about 14 days.

32. A method for managing serum estradiol concentration during a menstrual cycle in a female subject, the method comprising administering at least one estrogenic compound according to a regimen that comprises:
(a) a first regimen component wherein at least one estrogenic compound is administered to the subject according to a first mode of administration; said first mode of administration resulting in maintenance of a serum estradiol concentration of at least about 50 pg/ml during most or all of the menstrual cycle, except for an estrogen depleting phase of about 5 to about 10 days in duration beginning not earlier than about 7 days prior to menses; and
(b) a second regimen component wherein at least one estrogenic compound is administered to the subject according to a second mode of administration; said second mode of administration (i) comprising administering to a vulvovaginal surface a pharmaceutical composition that is bioadhesive thereto and comprises said at least one estrogenic compound; and (ii) resulting in moderation of a decline in serum estradiol concentration during said estrogen depleting phase.

33. The method of claim 32, wherein the first mode of administration is by one or more routes selected from the group consisting of oral, transdermal, parenteral, subcutaneous, intravenous, intramuscular, intraperitoneal, transmucosal, buccal, sublingual, intravaginal, inhalation, depot injection, implantation and combinations thereof.

34. The method of claim 32, wherein the first regimen component is an element of an oral contraceptive regimen.

35. The method of claim 32, wherein the decline in serum estradiol concentration in the absence of the second regimen component would be precipitous.

36. The method of claim 35, wherein the precipitous decline in serum estradiol concentration in the absence of the second regimen component would be conducive to a migrainous disorder.

37. The method of claim 32, wherein the pharmaceutical composition administered according to the second regimen component is a vaginal cream having at least one nonlipoidal internal phase and at least one lipoidal external phase that is bioadhesive to a vaginal mucosal surface, wherein the at least one estrogenic compound is present in an amount of about 50 μg to about 500 μg estradiol equivalent per unit dose of the vaginal cream, and wherein upon application of the composition to the vaginal mucosal surface the at least one estrogenic compound is released over a period of about 3 hours to about 14 days.

38. A kit for managing serum estradiol concentration during a menstrual cycle in a female subject, the kit comprising:
(a) a first regimen component wherein at least one estrogenic compound is administered to the subject according to a first mode of administration; said first mode of administration resulting in maintenance of a serum estradiol concentration of at least about 50 pg/ml during most or all of the menstrual cycle, except for an estrogen depleting phase of about 5 to about 10 days in duration beginning not earlier than about 7 days prior to menses; and
(b) a second regimen component wherein at least one estrogenic compound is administered to the subject according to a second mode of administration; said second mode of administration (i) comprising administering to a vulvovaginal surface a pharmaceutical composition that is bioadhesive thereto and comprises said at least one estrogenic compound; and (ii) resulting in moderation of a decline in serum estradiol concentration during said estrogen depleting phase.

39. The kit of claim 38, wherein said regimen comprises administration of the composition(s) by one or more routes selected from the group consisting of oral, transdermal, parenteral, subcutaneous, intravenous, intramuscular, intraperitoneal, transmucosal, buccal, sublingual, intravaginal, inhalation, depot injection, implantation and combinations thereof.

40. The kit of claim 38, wherein said regimen is an oral contraceptive regimen.

41. The kit of claim 38, further comprising instructions for administration of the vaginal cream to prevent or treat a catamenial migrainous disorder promoted by said regimen.

42. The kit of claim 38, further comprising an ovulation indicator.

43. The kit of claim 38, wherein the pharmaceutical composition administered according to the second regimen component is a vaginal cream having at least one nonlipoidal internal phase and at least one lipoidal external phase that is bioadhesive to a vaginal mucosal surface, wherein the at least one estrogenic compound is present in an amount of about 50 μg to about 500 μg estradiol equivalent per unit dose of the vaginal cream, and wherein upon application of the composition to the vaginal mucosal surface the at least one estrogenic compound is released over a period of about 3 hours to about 14 days.
